(19) United States
(12) Patent Application Publication
(10) Pub. No.: US 2025/0295546 A1
(43) Pub. Date: Sep. 25, 2025

(54) DEVICE FOR BILATERAL REHABILITATION

(71) Applicant: CONSIGLIO NAZIONALE DELLE RICERCHE, Roma (IT)

(72) Inventors: Matteo MALOSIO, Roma (IT); Matteo LAVIT NICORA, Roma (IT); Davide REDAELLI, Roma (IT); Giovanni TAURO, Roma (IT)

(21) Appl. No.: 18/863,189

(22) PCT Filed: May 4, 2023
(86) PCT No.: PCT/IB2023/054653

§ 371 Date: Nov. 5, 2024

(30) Foreign Application Priority Data
May 6, 2022 (IT) 102022000009317

(51) Int. Cl. A61H 1/02; G16H 40/63

(52) U.S. Cl. CPC A61H 1/0274; G16H 40/63; A61H 2201/10; A61H 2201/1215; A61H 2201/149; A61H 2201/1635; A61H 2201/5007; A61H 2201/5038; A61H 2201/5064; A61H 2230/045; A61H 2230/085; A61H 2230/105

(57) ABSTRACT

A device for bilateral rehabilitation of a user's limbs, includes a main body connected to a base, a first motor and a second motor supported by the main body, a first and second rotary interfaces supported by the main body rotatably operable about first and second rotation axes by the motors. Grip accessories have a grip portion and a coupling portion couplable to the first and second rotary interfaces. An electronic control device is configured to control rotary movements and torques applied to the first and second rotary interfaces depending on angular positions or torques detected by a position or torque detector. The main body is connected to the base by an adjustable and lockable joint allowing adjustment and locking of the position of the main body with respect to the base in at least three different body positions with different orientations of the first and second rotation axes.

### CROSS-REFERENCE TO RELATED APPLICATIONS

The present application is a National Stage Filing of PCT International Application No. PCT/IB2023/054653 filed on May 4, 2023, which claims priority to Italian application No. 102022000009317, filed with the Italian Patent Office on May 6, 2022, which applications are incorporated herein by reference in their entirety.

### FIELD OF THE INVENTION

The present invention relates to a bilateral rehabilitation device for the upper and/or lower limbs of a user (patient) by means of the interaction between the user and the device for carrying out various motor gestures of the user's upper or lower limbs, involving different joints, in order to contribute to the recovery of the functionality and motor coordination of the upper or lower limbs and to facilitate a so-called cerebral plasticity mechanism.

### SUMMARY OF THE INVENTION

Bilateral rehabilitation of the upper and lower limbs is based on the assumption that during coordinated bilateral movements, the movement of the non-paretic limb can support the stimulation of the movement of the paretic limb at the cerebral level. Such a rehabilitation approach can be effective for example in cases of cerebral palsy, stroke, or other cases of hemiparesis.

Bilateral rehabilitation devices, especially those intended for autonomous use in the home, should ideally meet the following requirements: portability, structural and user simplicity (ergonomics), adaptability to different anthropometric sizes, versatility for carrying out different types of exercises (monolateral/bilateral), low costs.

The availability of increasingly low-cost mechatronic technologies on the one hand, and the growing demand for rehabilitation solutions and equipment for out-of-hospital and home use, in order to intensify patient care, on the other hand, are stimulating the research and development of innovative rehabilitation equipment which reconciles the need to limit costs with the need for versatility of use.

However, the rehabilitation devices currently on the market do not allow use in multiple different configurations. Therefore, rehabilitation plans with different motor gestures require the purchase and use of multiple specific devices. It would instead be desirable to have a single device capable of supporting multiple different motor gestures.

The bimanual rehabilitation devices listed below are known to the inventors and can be considered a background art for the present invention.

The BATRAC® device and Rocker® device are non-configurable manual rehabilitation devices which allow only one movement of the hands, but not also of other joints of the entire upper limb, for example the shoulder, elbow.

Other devices known to the inventors or under development allow wider movement of the upper limb, but constrain the movements of the upper limbs along fixed, unchangeable directions.

The Tailwind® device is a so-called “passive”, non-motorized device which determines the movement of the upper limbs by means of two linear guides.

The Reha-Slide Duo® device is similar to the Tailwind® device and does not allow orienting the movement directions of the guides in an independent manner, but allows mutually constraining the movements of the two manipulandums.

The Bimanual Lifting Rehabilitator® device allows adjusting a lifting intensity depending on the movements and forces exchanged between the user's hands and interface devices (handles), amplifying the lifting/raising force. However, even the Bimanual Lifting Rehabilitator® device does not allow any reconfigurations to carry out multiple different motor gestures.

Rehabilitation devices with accessories for guiding and carrying out movements with two upper limbs, such as the Iron Arm, are also known, but the possible movements are limited in one plane and independent movements of the two upper limbs are not possible.

Devices are also known (Hand Robotic Rehabilitation Device®) with two motorized cranks with adjustable rotation axes, structurally divided into several separate housings and therefore classifiable as a system consisting of several distinct devices with the related installation dimensions.

More complex bimanual rehabilitation devices are also known, for example bilateral exoskeletons, for example EXO-UL7®, which allow a series of different motor gestures but require a vertical wall installation in the hospital.

Therefore, the purpose of the present invention is to provide a device for the bilateral rehabilitation of the limbs (understood as upper and/or lower limbs) having features such as to be usable also in the domestic field, structurally simple, less cumbersome with respect to the devices of the known art, as well as configurable for carrying out different types of movement.

### BRIEF DESCRIPTION OF THE DRAWINGS

In order to better understand the invention and appreciate the advantages thereof, some non-limiting embodiments thereof will be described below by way of non-limiting example with reference to the accompanying drawings, in which:

FIGS. 1, 2, 3 are perspective views of a bilateral rehabilitation device according to an embodiment,

FIGS. 4, 5, 6, 7 are front (FIG. 4), bottom (FIG. 5), side (FIGS. 6, 7) views of the rehabilitation device in FIG. 1,

FIGS. 8A, 8B, 8C are front views of the rehabilitation device in three different rotary axis orientation configurations, according to an embodiment,

FIG. 9 is an exploded view of the rehabilitation device, according to an embodiment,

FIGS. 10.1 and 10.2 are perspective views of the rehabilitation device with a first set of grip accessories in two different gripping orientation configurations, such accessories being suitable for both the upper limbs and the lower limbs,

FIGS. 10.3 and 10.4 are perspective views of the rehabilitation device with a second set of grip accessories in two different gripping orientation configurations and in two different rotation axis orientation configurations, such accessories being suitable for both the upper limbs and the lower limbs,

FIG. 10.5 is a perspective view of the rehabilitation device with a third set of grip accessories (cranks, pedals) in a gripping orientation configuration of a plurality of gripping orientation configurations, and in a rotation axis configuration of a plurality of different rotation axis orientation configurations, such accessories being suitable for both the upper limbs and the lower limbs,

FIG. 10.6 is a perspective view of the rehabilitation device with a fourth set of grip accessories (linkage) in a gripping orientation configuration of a plurality of gripping orientation configurations, and in a rotation axis configuration of a plurality of different rotation axis orientation configurations,

FIG. 10.7 is a perspective view of the rehabilitation device with a fourth set of grip accessories similar to the third set of handle accessories (cranks, pedals) in a different gripping orientation configuration, and with an electronic screen, such accessories being suitable for both the upper limbs and the lower limbs,

FIG. 10.8 is a perspective view of the rehabilitation device with a further set of mutually different grip accessories, in a reconfigurable gripping orientation configuration, and with an electronic screen, such accessories being suitable for both the upper limbs and the lower limbs,

FIG. 10.9 is a diagrammatic view of a translational grip accessory, which is operable by a rotary-translational motion conversion mechanism, such an accessory being suitable for both the upper limbs and the lower limbs,

FIG. 11 is an exploded view of a mechanism for adjusting the orientation of a grip accessory of the rehabilitation device, according to an embodiment,

FIG. 12 shows a motorized drive mechanism of the rehabilitation device according to an embodiment,

FIG. 13 is a partially exploded view of a portion of the motorized drive mechanism in FIG. 12,

FIGS. 14.1, 14.2, 14.3, 14.4, 14.6 show control block diagrams of the bilateral rehabilitation device according to embodiments, in which:

FIG. 14.1 shows an angular speed estimator module,

FIG. 14.2 shows an angular speed control module

FIG. 14.3 shows an angular position control module

FIG. 14.4 shows an admittance control module,

FIG. 14.5 shows a bimanual (or similarly bi-foot or generically bi-arm or bilateral) virtual mechanics model,

FIG. 14.6 shows a bi-lateral impedance control module implementing the virtual mechanics model of FIG. 14.5,

FIG. 15 is a block diagram of an operating method of the rehabilitation device according to an embodiment.

### DETAILED DESCRIPTION OF THE INVENTION

According to an aspect of the invention, a bilateral rehabilitation device 1 for a user's limbs (understood as upper or lower) comprises:
a connecting base 2 with connecting members 3 for a stationary connection of the connecting base 2 to a support 4,
a main body 5 connected to the connecting base 2,
a first electric motor 7′ and a second electric motor 7″ supported by the main body 5,
a first rotary interface 8′ supported by the main body 5 in a first position and which is rotatably operable about a first rotation axis 15′ by the first motor 7′, and a second rotary interface 8″ supported by the main body 5 in a second position spaced from the first position and rotatably operable about a second rotation axis 15″ by the second motor 7″,
a set 12 of grip accessories 12.1, 12.2, . . . , 12.n each having a grip portion 13 (intended to be gripped by the user) and a coupling portion 14 which is reversibly couplable integral in rotation to one of the first 8′ and second 8″ rotary interfaces, respectively,
an electronic control device 11 in signal connection with the first motor 7′ and with the second motor 7″ and comprising:
first position detecting means 9′ for detecting an angular position of the first rotary interface 8′ with respect to the main body 5,
second position detecting means 9″ for detecting an angular position of the second rotary interface 8″ with respect to the main body 5,
first torque detecting means 10′ for detecting a torque applied to the first rotary interface 8′,
second torque detecting means 10″ for detecting a torque applied to the second rotary interface 8″,
in which the electronic control device 11 is configured to control the rotary movements and torques applied to the first 8′ and second 8″ rotary interfaces depending on the angular positions and/or the torques detected by the first and second position detecting means 9′, 9″ and the first and second torque detecting means 10′, 10″,
in which the main body 5 is connected to the connecting base 2 by means of an adjustable and lockable joint 6 which allows an adjustment and locking of the position of the main body 5 with respect to the connecting base 2 in at least three (or at least two or three) different body positions with different orientations of the first 15′ and second 15″ rotation axes.

In this context the feature “different body positions with different orientations of the first 15′ and second 15″ rotation axes” means that the orientation of the first rotation axis 15′ changes when the body position changes and also the orientation of the second rotation axis 15″ changes when the body position changes. Instead, the orientation of the first rotation axis 15′ with respect to the orientation of the second rotation axis 15″ does not necessarily change. Both the first 15′ and second 15″ rotation axes can maintain, for example, a mutually fixed relative position or can be parallel or coaxial, i.e., coincident.

By virtue of the configuration of the rehabilitation device 1, it can be used in a domestic environment, structurally simple and compact, as well as configurable for carrying out different types of limb movement.

The adjustment of position and orientation of two rotation axes of two different spaced apart rotary interfaces 8′, 8″ by means of a position adjustment of a single main body 5 supporting both motors 7′, 7″ and both rotary interfaces 8′, 8″ makes the rehabilitation device 1 particularly versatile and suitable for a high number of different motor gestures for each arm and combinations of motor gestures of both limbs, for example upper, synchronized, asynchronous, dependent, independent, symmetrical, non-symmetrical, in the same direction or in the opposite direction, simultaneous or in succession.

The possibilities of defining free movements adapted to the conditions of the individual patient are further increased by virtue of the set 12 of grip accessories 12.1, 12.2, . . . , 12.n which are couplable to the rotary interfaces 8′, 8″.

### Detailed Description of the Connecting Base 2

According to an embodiment, the connecting base 2 can comprise a “U” or clamp shape defining a connecting seat 16 which is laterally accessible for receiving for example an edge of a table (support 4).

The connecting members 3 can comprise, for example, clamps and/or tightening screws and/or adhesive surfaces and/or suction cups and/or magnets and/or Velcro, and allow a stationary, but removable, reversible and repositionable connection of the connecting base 2 to the support 4.

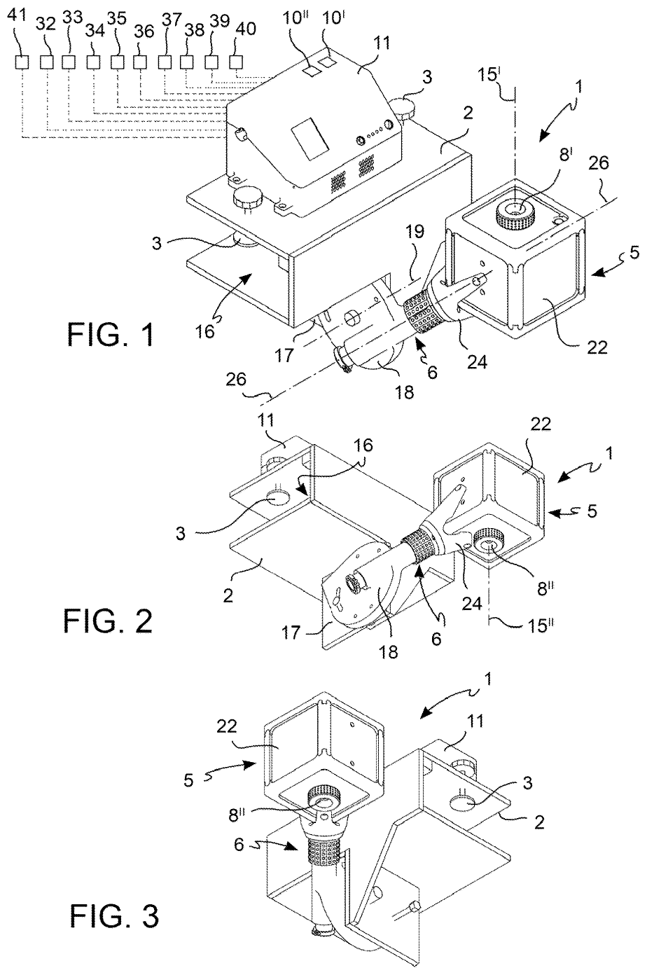

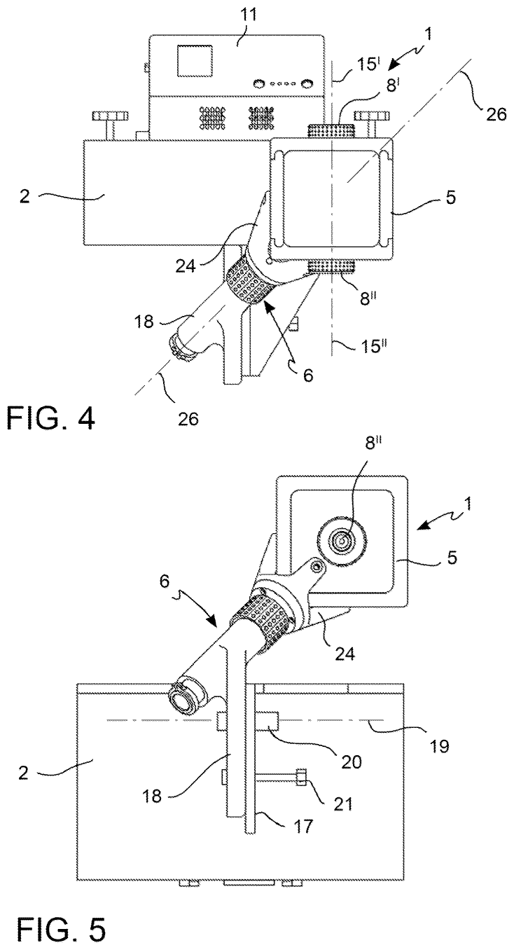

The connecting base 2 further forms a joint fastening portion 17 for fastening a base portion 18 of the adjustable joint 6 to the connecting base 2, for example for a fastening with the possibility of rotary adjustment of the position of the base portion 18 with respect to the connecting base 2, about an auxiliary adjustment axis 19, for example horizontal (FIGS. 1, 5, 6).

According to an embodiment, the base portion 18 and the joint fastening portion 17 can be connected to each other by means of a rotation pin 20 which constrains the adjustment movement thereof to rotation only about the auxiliary adjustment axis 19 and by means of a tightening bolt 21 inserted in a hole of one of the base portion 18 and the joint fastening portion 17, and in an arcuate groove of the other of the base portion 18 and the joint fastening portion 17 (FIGS. 2, 5).

### Detailed Description of the Main Body 5

In accordance with an embodiment, the main body 5 has an outer housing 22, for example in the form of a parallelepiped or a cube or a sphere, which accommodates, at least partially but preferably completely, the first motor 7′ and the second motor 7″ therein and, if provided, a first transmission 23′ (preferably with constant transmission ratio, preferably less than 1, i.e., a reduction ratio) connected between the first motor 7′ and the first rotary interface 8′, and a second transmission 23″ (preferably with constant transmission ratio, preferably less than 1, i.e., a reduction ratio) connected between the second motor 7″ and the second rotary interface 8″ (FIG. 12).

The first transmission 23′ and/or the second transmission 23″ can comprise, for example, a reduction gear or a belt and pulley transmission, for example reduction, or another similar and equivalent transmission system.

According to an embodiment, in order to reduce the overall dimensions of the main body 5 as much as possible, the first motor 7′ and the second motor 7″ are arranged parallel to each other and placed side by side, with the axial extensions thereof overlapping, so that the axial dimensions of both the first 7′ and second 7″ motors is smaller than the sum of the individual axial extensions thereof.

Still in order to reduce the overall dimensions of the main body 5 as much as possible, a first operating connection of the first motor 7′ to the first rotary interface 8′ (for example by means of the first transmission 23′ or by means of “direct drive” connection) and a second operating connection of the second motor 7″ to the second rotary interface 8″ (for example by means of the second transmission 23″ or by means of “direct drive” connection) are made/positioned on two opposite sides of the first 7′ and second 7″ motors or on two opposite sides of the main body 5. Preferably, the first transmission 23′ is positioned laterally next to the second motor 7″ with overlapping of the axial extensions thereof, and the second transmission 23″ is positioned laterally next to the first motor 7′ with overlapping of the axial extensions thereof, such that the total axial dimensions of both the first motor 7′+first transmission 21′ and second motor 7″+second transmission 21″ assemblies is identical to the individual axial dimensions of only one of the two first motor 7′+first transmission 21′ and second motor 7″+second transmission 21″ assemblies.

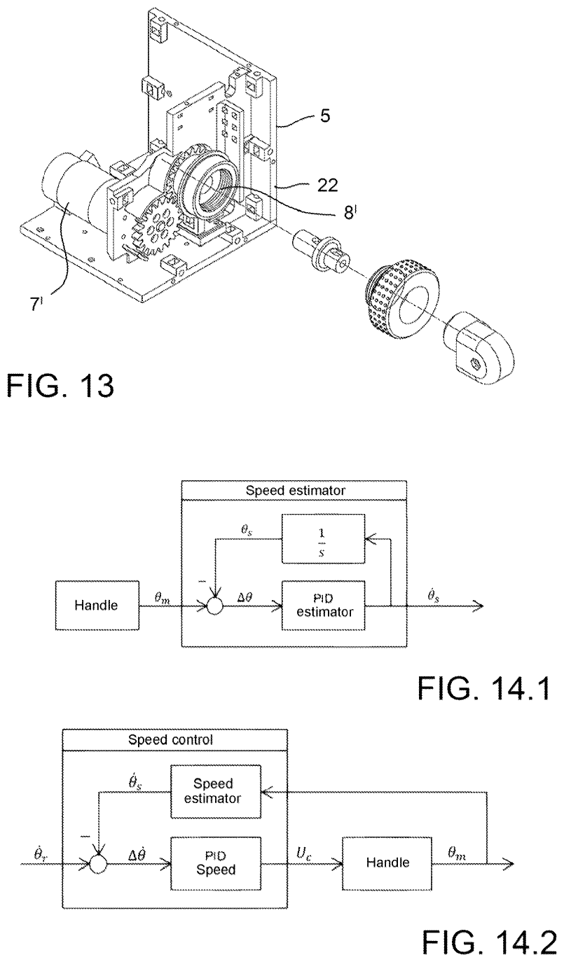

With further advantage, the first rotary interface 8′ and the second rotary interface 8″ are both in laterally displaced positions with respect to both the first motor 7′ and the second motor 7″. This avoids the accumulation of components one behind the other in the axial direction of the motors 7′, 7″, allowing a minimization of the dimensions of the main body 5 in the axial direction of the motors 7′, 7″ (FIGS. 12, 13).

In accordance with an embodiment, the first 8′ and second 8″ rotary interfaces are accessible from the outside of the main body 5 and preferably facing or turned towards two opposite directions and towards the outside of the main body 5.

Preferably, the first 8′ and second 8″ rotary interfaces are positioned on two opposite sides of the main body 5 and the first rotation axis 15′ and the second rotation axis 15″ are parallel, preferably coincident and coaxial.

The first 8′ and second 8″ rotary interfaces comprise quick coupling means, without the aid of tools, for example an elastic snap-fit coupler, a threaded ring nut coupler, a bayonet coupler, an insertion coupler or a threaded coupler, for a rotatably integral connection between the rotary interface 8′, 8″ and the coupling portion 14 of the grip accessory 12.1, 12.2, . . . , 12.n.

This allows a quick and easy reconfiguration of the rehabilitation device 1, by replacing and modifying the grip accessories 12.1, . . . , 12.n, even in the domestic environment and without the help of specialized personnel.

According to an embodiment, the adjustable joint 6 is configured so that the main body 5 is rotatable with respect to the connecting base 2 about a main adjustment axis 26 and lockable with respect to the connecting base 2, in which the main adjustment axis 26 is inclined with respect to the first rotation axis 15′ and with respect to the second rotation axis 15″.

The adjustable joint 6 can comprise an adjustable portion 24, integral with the main body 5, and rotatable with respect to the base portion 18 about a main adjustment axis 26 and lockable with respect to the base portion 18, in which the main adjustment axis 26 is inclined with respect to the first rotation axis 15′ and with respect to the second rotation axis 15″.

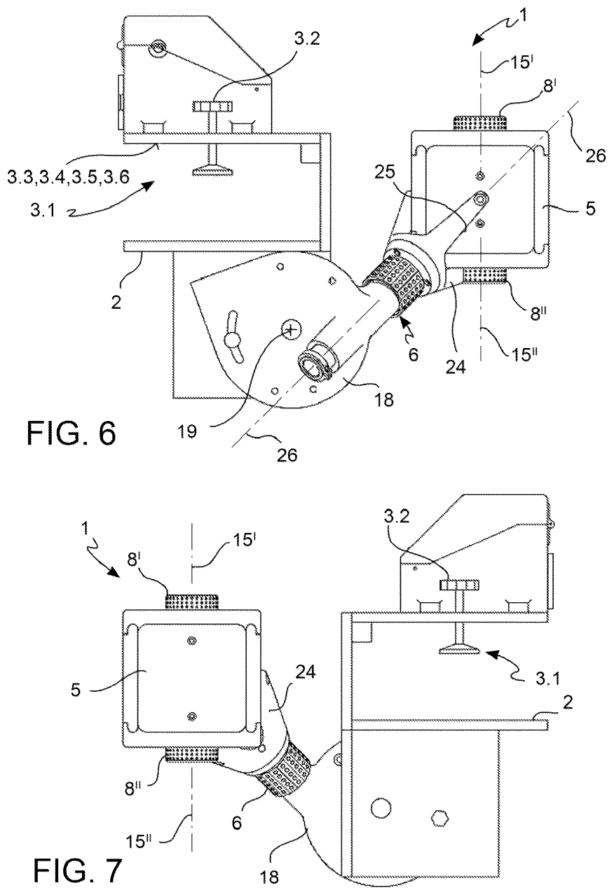

The adjustable portion 24 can be connected to the base body 5 in a fixed manner (not removable) or preferably, detachably (for example by screws) to a special support seat 25 of the main body 5 (FIGS. 6, 9). The detachable connection allows a storage and transport of the partially disassembled device 1 with particularly small dimensions and little packaging material.

By virtue of the inclination of the main adjustment axis with respect to the first rotation axis 15′ and the second rotation axis 15″, the latter can be oriented in the aforesaid at least three body positions in three mutually transverse, preferably orthogonal, directions.

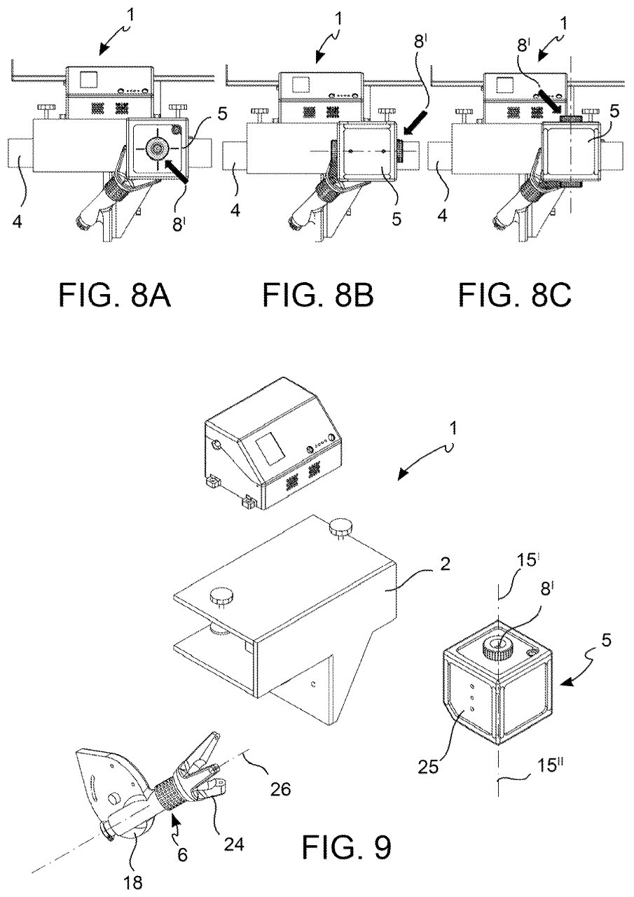

More precisely, the main adjustment axis 26 is inclined with respect to (and preferably but not necessarily incident with) the rotation axes 15′, 15″ in turn coinciding with each other, at an angle in the range between 45° and 65°, preferably between 50° and 60°, more precisely at an angle arctan (Sqrt (2)), i.e., 54.74°. This allows the orientation of the first 15′ and second 15″ rotation axes in the space along mutually orthogonal directions (in FIGS. 8A, 8B, 8C the same rotary interface is indicated by an arrow to show the three orthogonal orientations).

For a cube shape of the main body 5 with the first rotation axis 15′ and the second rotation axis 15″ coinciding and centered on two opposite faces of the cube, the inclination angle between the main adjustment axis 26 or the first and second rotation axes 15′, 15″ is equal to the angle formed by the diagonal of the cube with the normal angle at each of its faces, equal to arctan (Sqrt (2)).

### Detailed Description of the Set 12 of Grip Accessories 12.1, . . . , 12.n

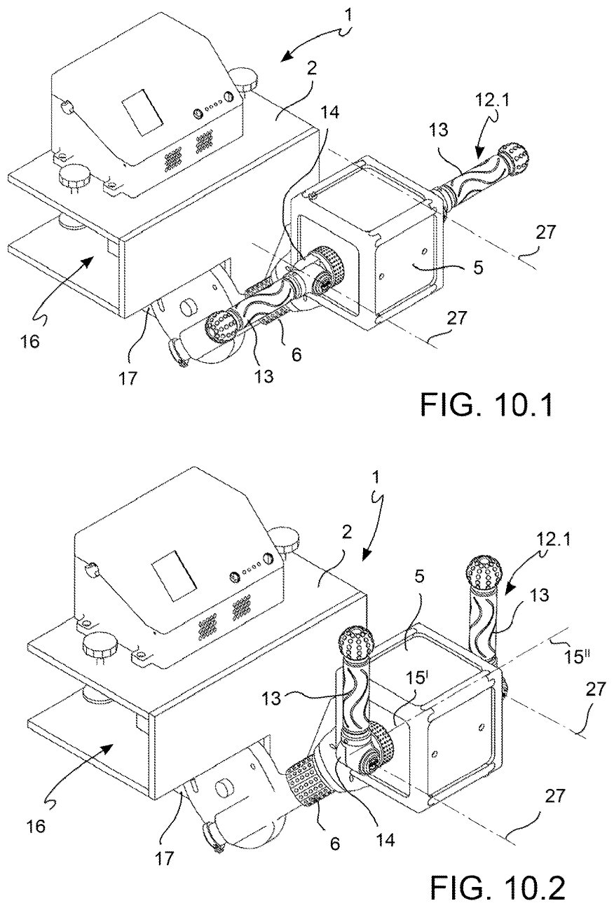

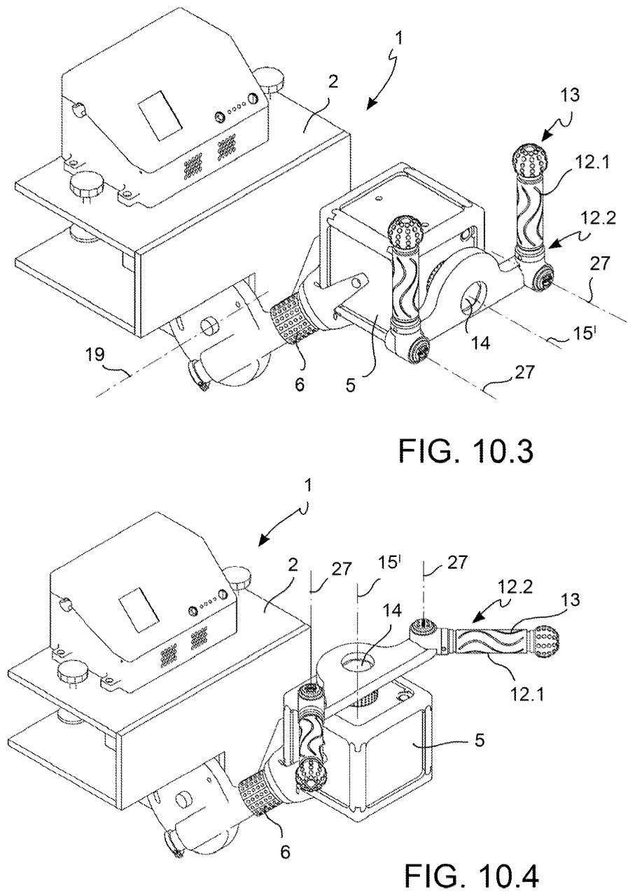

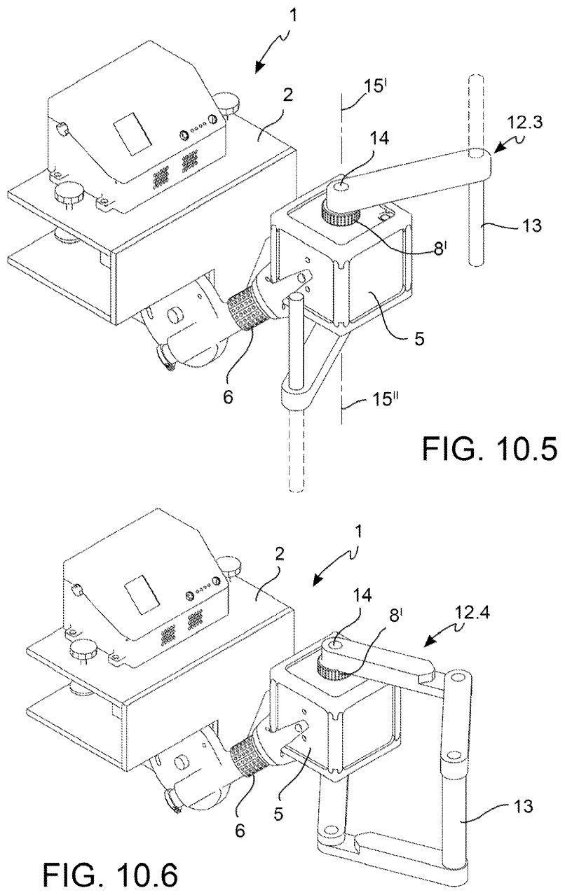

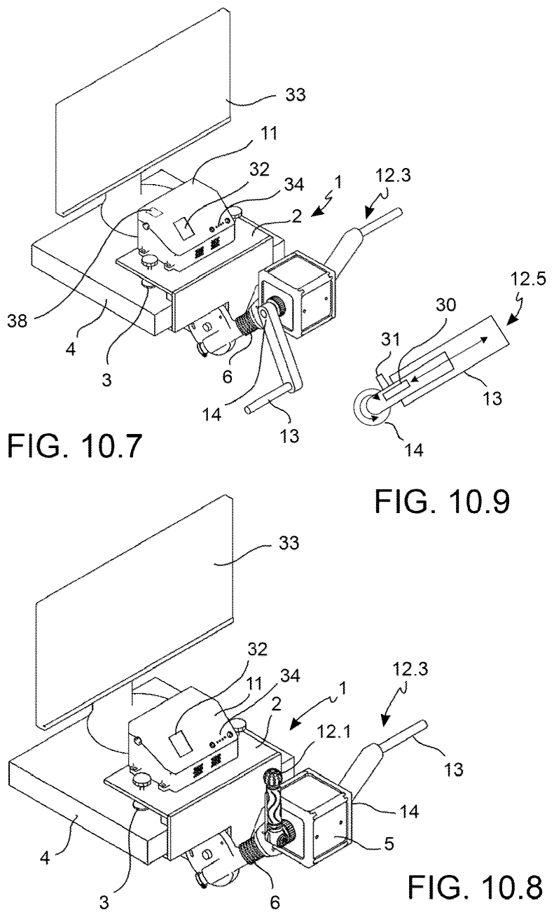

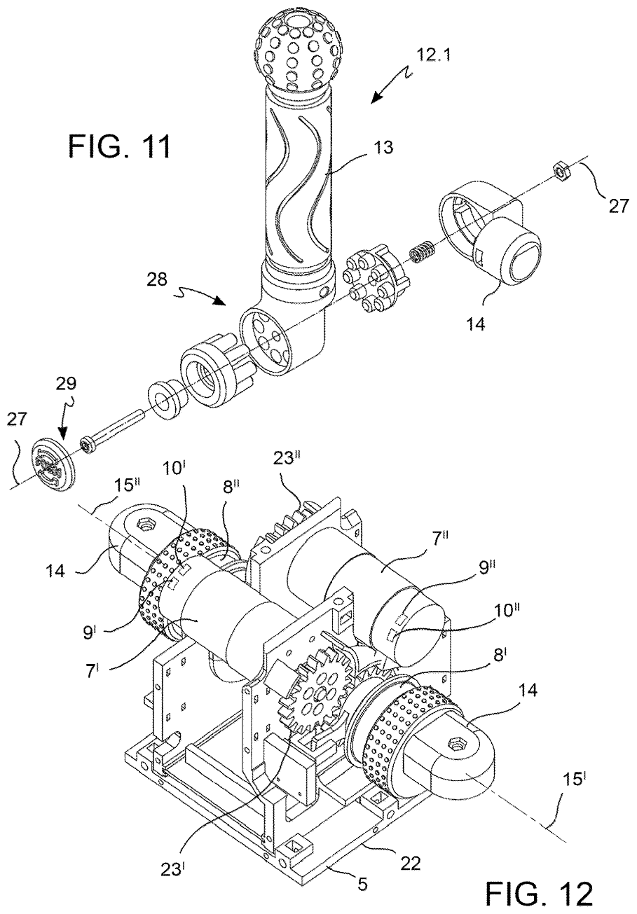

In accordance with an embodiment, the grip accessories 12.1, . . . , 12.n comprise two handlebar accessories 12.1 (FIGS. 10.1, 10.2) each couplable to one of the first 8′ and second 8″ rotary interfaces, respectively, in which the grip portion 13 is orientable (and lockable in position) with respect to the coupling portion 14, about a handlebar orientation axis 27 transverse (preferably orthogonal) to the first rotation axis 15′ and to the second rotation axis 15″.

To this end, the handlebar accessory 12.1 can comprise an orientation and locking mechanism 28 (FIG. 11), for example elastically lockable and releasable by pressing a button 29.

In accordance with an embodiment, the grip accessories 12.1, . . . , 12.n comprise a wheel accessory 12.2 (FIGS. 10.3, 10.4) couplable to one of the first 8′ and second 8″ rotary interfaces, and comprising two grip portions 13 spaced apart from each other and positioned on two opposite sides with respect to the first 8′ or second 8″ rotary interface, and adjustable (and lockable in position) with respect to the coupling portion 14, about respective handlebar orientation axes 27, 27 for example parallel to the first rotation axis 15′ or to the second rotation axis 15″.

To this end, the wheel accessory 12.2 can comprise for each grip portion 13 an orientation and locking mechanism 28 (FIG. 11), for example elastically lockable and releasable by pressing a button 29.

In accordance with an embodiment, the grip accessories 12.1, . . . , 12.n comprise two crank accessories 12.3 (FIG. 10.5) each couplable to one of the first 8′ and second 8″ rotary interfaces, respectively, in which the grip portion 13 is parallel and spaced with respect to the first rotation axis 15′ and the second rotation axis 15″. The coupling portion 14 of the crank accessories 12.3 can be configured for coupling with the respective rotary interface 8′, 8″ in two different positions of the crank accessory 12.3, mutually overturned by 180° (FIG. 10.5).

In accordance with an embodiment, the grip accessories 12.1, . . . , 12.n comprise a linkage accessory 12.4 or articulated multilateral accessory (FIG. 10.6) having two coupling portions 14 both connected to a single grip portion 13 by means of two articulated linkages.

In accordance with an embodiment, the grip accessories 12.1, . . . , 12.n comprise a translational accessory 12.5 (FIG. 10.9), in which the grip portion 13 is linearly translatable with respect to the coupling portion 14, and which comprises a non-self locking, for example rack, motion conversion mechanism 30 interposed between the grip portion 13 and the coupling portion 14, as well as anti-rotation means 31 which prevent a rotation of the grip portion 13 with respect to the main body 5.

FIG. 10.8 shows an embodiment in which different grip accessories 12.1, . . . , 12.n are connected to the main body 5.

The coupling portions 14 of the grip accessories 12.1, . . . , 12.n have a sure angular reference for a connection with the rotary interface 8′, 8″ in a single angular position, allowing the electronic control device 11 to always know the spatial orientation thereof in a unique manner.

### Detailed Description of the Electronic Control Device 11

The control device 11 can comprise a computer as shown in the figures, fastened for example on the connecting base 2, and provided with or connectable to a user interface 32 with a display 33 and/or a keyboard 34. Alternatively, the control device 11 can be inside the main body 5. In an embodiment, the control device 11 can be connected or connectable (by means of Wi-Fi, Bluetooth, etc.) to an external electronic device (e.g., a tablet computer or a smart phone) serving as a user interface.

The control device 11 is configured to allow the selection of a plurality of rehabilitation programs/modes with the possibility of adjusting a plurality of movement parameters of the respective rehabilitation program/mode.

The rehabilitation programs/modes comprise rehabilitation programs with synchronized movements of the first rotary interface 8′ and the second rotary interface 8″ and rehabilitation programs with movements of the first rotary interface 8′ and the second rotary interface 8″ which are mutually different.

In accordance with an embodiment, the control device 11 is configured to perform rehabilitation programs with a control of the first motor 7′ and the second motor 7″ by means of a control in position and in rotary speed, particularly suitable for passive rehabilitation, in which the rehabilitation device 1 imposes a predefined movement on the user's limbs.

In accordance with an additional or alternative embodiment, the control device 11 is (further) configured to execute rehabilitation programs with a control of the first motor 7′ and the second motor 7″ by means of an impedance and/or input control, based on:
movements/positions of the first rotary interface 8′ and of the second rotary interface 8″ detected by the first position detecting means 9′ and second position detecting means 9″,
theoretical movements/positions of the first rotary interface 8′ and of the second rotary interface 8″ commanded by the control device 11, and
the corresponding torques applied/transmitted to the first rotary interface 8′ and to the second rotary interface 8″, detected by the first torque detecting means 10′ and second torque detecting means 10″.

The impedance/admittance control is particularly suitable for a motor rehabilitation in which movements carried out by the user themselves are supported to the extent necessary (“assist-as-needed”) so as to support the (residual) motor skills of the user based on his real residual abilities at the time of use of the device 1.

According to an embodiment, the control device 11 is configured or configurable for a control of the movement of the first motor 7′ and the second motor 7″ in a coordinated and dependent manner, according to one or more dependency parameters and/or one or more dependency functions, for example predefined or selectable by the user interface 32.

The dependency parameters or functions of the movements of the first motor 7′ and of the second motor 7″ can be selectable so that the second motor 7″ is controlled depending on a detected movement of the first rotary interface 8′, so as to impose a movement of the second rotary interface 8″:
in time offset with respect to the movement of the first rotary interface 8′ by a time offset value (selectable as a function of rehabilitation needs), and/or
in position offset, forward or backward, with respect to the movement of the first rotary interface 8′ by a position offset value (selectable as a function of rehabilitation needs), and/or
in the opposite direction with respect to the movement of the first rotary interface 8′ with or without time offset and/or position offset.

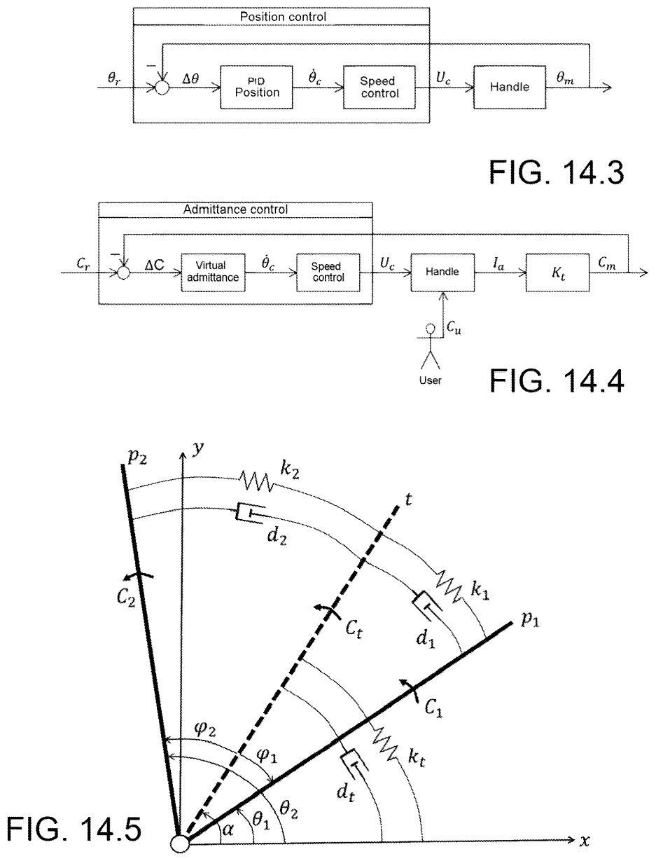

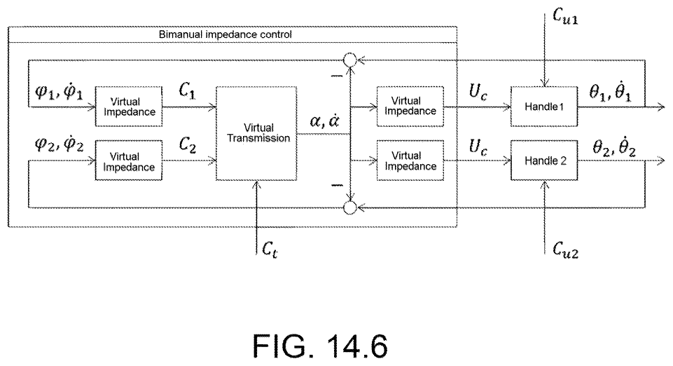

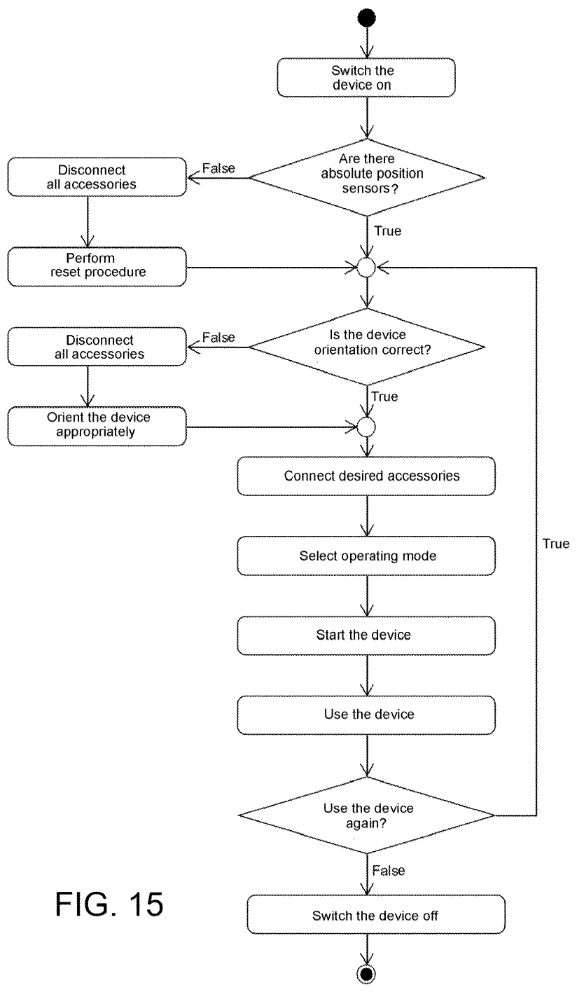

In accordance with an embodiment, the first and second position detecting means 9′, 9″ comprise an encoder for each of the first 8′ and second 8″ rotary interfaces (such encoders can preferably be placed directly on the first 7′ and second 7″ motors), but possibly no further sensor for the direct measurement of rotational speeds, or alternatively a first rotary speed sensor for detecting the rotary speed of the first rotary interface 8′ and a second rotary speed sensor for detecting the rotary speed of the second rotary interface 8″ could be provided. The angular position detection can be redundant (two encoders or angular position detectors associated with each of the two rotary interfaces 8′, 8″) with consistency check, with angular position detection incrementally and/or absolutely, so as to avoid a homing procedure (initial reset of the starting angular position). The rotational speed value can be estimated by the control device 1, using a closed-loop logic, as depicted in FIG. 14.1. The angular position measure θₘ of each rotary interface 8′, 8″ is compared with the estimated angular position value θₛ starting from the current rotational speed estimate \dot{θ}₅. The calculated error Δθ is then used within a PID logic (Proportional-Integrative-Derivative, or similar processing module) which outputs the rotational speed value estimate for the next control cycle.

In accordance with an embodiment, using the speed estimation method described above, the control device 11 can be configured to implement a second closed loop for controlling the first rotary interface 8′ and the second rotary interface 8″ in speed as shown in FIG. 14.2. The error between the reference rotational speed \dot{θ}ᵣ and the actually estimated rotational speed \dot{θ}ₛ starting from the measured position θₘ is used within a PID logic (or analogous processing module) which outputs the motor command U_{c} for the motor 7′, 7″ for the corresponding rotary interface 8′, 8″.

In order to ensure the safety of the user, the control device 11 continuously monitors the current absorbed by the motors 7′, 7″ and the rotational speeds and torques transmitted by means of the first 8′ and second 8″ rotary interfaces, verifying that appropriate imposed limits are not exceeded.

According to an embodiment, the first torque detecting means 10′ and the second torque detecting means 10″ can comprise means for detecting the current absorbed by the first motor 7′ and the second motor 7″, respectively, and means for estimating/determining the transmitted torque as a function of the detected absorbed current.

According to an embodiment, the first torque detecting means 10′ and the second torque detecting means 10″ can comprise one or more load cells, for example connected to the motor 7′, 7″ or to the rotary interface 8′, 8″, and configured to provide a signal representative of the torque transmitted respectively to the/from the first rotary interface 8′ and/or the second rotary interface 8″.

Similarly, the closed loop for controlling each of the first 8′ and second 8″ rotary interfaces in position is shown in FIG. 14.3. The error between the reference angular position θᵣ and the currently measured angular position θₘ is used within a PID logic (or a processing/adjustment module other than PID) to generate a speed command \dot{θ}_{c}. for the motor 7′, 7″. The new rotational speed reference is then input to the rotational speed control cycle previously described (FIG. 14.2) to generate the actual motor command U_{c}.

According to an embodiment, the control device 11 is configured to monitor the tracking error (i.e., the difference between the position value and/or actual angular speed, detected, and the commanded target value thereof) to implement a fail safe operation.

According to an embodiment, with the aim of controlling both the angular position and the moment (torque) transmitted by each of the first 8′ and second 8″ rotary interfaces, it is advantageous to implement by means of the control device 11 (all the method algorithms described herein are advantageously executed by the control device 11 which is thus configured to execute them) an admittance control loop, shown for example in FIG. 14.4. The control device 11 makes an estimate of the torque Cₘ exerted by each of the first 7′ and second 7″ motors (for example proportional to the absorbed current Iₐ by means of the proportionality coefficient Kₜ) and compares the estimated torque value Cₘ with the reference torque Cᵣ. The calculated difference in torque ΔC between the estimated torque and the reference torque is applied (“made to act”) on a virtual model with predefined admittance features. By virtue of this approach, it is possible to define a reference rotary speed \dot{θ}_{c} which is then input to the speed control previously described (FIG. 14.2) to obtain the actual command U_{c} at the first 8′ and second 8″ rotary interfaces, i.e., at the grip accessories 12.1, . . . , 12.n where a torque Cᵤ is exchanged with the user.

According to an embodiment, one or more or all of the described control patterns are applied to each of the first 8′ and second 8″ rotary interfaces of the device 1 in an independent manner.

Alternatively or (advantageously) additionally, the control device 11 is configured or settable by means of program selection by a user, so as to control the movements and the torques applied to the first 8′ and second 8″ rotary interfaces in a dependent manner, in order to obtain a bilateral (bi-limb) device-user interaction.

According to an embodiment, the movements and the torques applied to the first 8′ and second 8″ rotary interfaces, or in other words the first 8′ and second 8″ rotary interfaces, are (dynamically) linked to each other by the logic (dynamic model of motion and torque coupling) diagrammatically depicted in FIG. 14.5. In FIGS. 14.5, p₁ and p₂ indicate the two first 8′ and second 8″ rotary interfaces, while t represents a virtual transmission element capable of scaling the effect of the two first 8′ and second 8″ rotary interfaces by means of the scale parameters τ₁ and τ₂. As depicted, p₁ and p₂ are linked to t through virtual impedances characterized by stiffness and damping values respectively indicated with k₁, d₁ and k₂, d₂ and having preset, selectable and/or adjustable values. In turn, the virtual transmission element t is connected to the absolute reference xy by means of a spring-damper system (numerical model) characterized by the stiffness and damping values indicated with kₜ, dₜ and having preset, selectable and/or adjustable values. The angles formed by p₁, p₂ and t with the absolute reference system xy are indicated by θ₁, θ₂ and α respectively. Furthermore, φ₁ and φ₂ represent the relative angle measured between p₁, t and p₂, t respectively. The torques acting on p₁ and p₂ are indicated by C₁ and C₂, respectively, while Cₜ represents an external torque applied directly to t to assist the user's movement. Finally, Cᵤ₁ and Cᵤ₂ are the torques transmitted to the first 8′ and second 8″ rotary interfaces by the user's limbs.

By virtue of this approach, by modifying the described control parameters, it is possible both to adjust the torque intensity applied from the outside and the torque intensity which one limb is capable of transmitting to the other, and to scale the ranges of movement of the two limbs based on the user's residual mobility.

By exploiting and applying the method and dynamic model of bilateral dependence (bimanual or bi-foot) described, according to an embodiment the control device 11 is configured to implement a control method shown by way of example in FIG. 14.6.

For each of the first 8′ and second 8″ rotary interfaces, the control device calculates angular position and rotary speed based on the absolute angular position measurement as follows.

$${φ}_{1} = {θ}_{1} - α$$

$${φ}_{2} = {θ}_{2} - α$$

Depending on the rotary speed values, the control device 11 calculates the torques applied on the virtual impedances and then transmitted on the virtual transmission element.

$${C}_{1} = {k}_{1} {φ}_{1} + {d}_{1} {\dot{φ}}_{1}$$

$${C}_{2} = {k}_{2} {φ}_{2} + {d}_{2} {\dot{φ}}_{2}$$

Depending on the positions and angular speeds, as well as the torques applied on the virtual transmission element, the control device 11 solves the dynamic motion equations of the virtual transmission to extrapolate the measurements of a and à (angular position and angular speed) for the next control cycle.

$${k}_{t} α + {d}_{t} \dot{α} = {τ}_{1} {C}_{1} + {τ}_{2} {C}_{2} + {C}_{t}$$

The angular position and angular speed values (here synonymous with rotary speed) of the extrapolated virtual transmission element are used by the control device 11 as a basis for generating the actual power supply command U_{c} sent to each of the first 7′ and second 7″ motors.

In accordance with embodiments, the control device 11 is in signal communication with one or more of:
one or more electromyographic sensors 35,
one or more functional electrical stimulation devices 36,
one or more EEG signal detectors 37,
one or more electronic graphical and/or auditory user interfaces 38,
one or more EDA (electrodermal activity) sensors 39,
one or more ECG (electrocardiac activity) sensors 40,
one or more digital cameras 41 for monitoring the user and/or movements of the user's limbs,
and is configured to control the first motor 7′ and the second motor 7″ depending on signals provided thereby.

According to an embodiment, the first 8′ and second 8″ rotary interfaces comprise electrical contact means for transmitting electrical signals between the grip accessory 12.1, . . . , 12.n and the control device 11, for example for transmitting signals generated by one or more force or pressure sensors connected to the grip portion 13, and/or for transmitting activation signals for one or more light signals (LEDs) connected to the grip portion 13.

In the described examples the motors 7′, 7″ are electric motors, but they can also be different motors, for example pneumatic motors.

The rehabilitation device 1 allows carrying out different motor gestures in bilateral rehabilitation activities, capable of involving the joints of the limb, for example upper, in a different manner. This is possible by virtue of the possibility of using different grip accessories 12.1, . . . , 12.n, in combination with the possibility of versatile orientation of the main body 5 and the relative rotation axes 15′, 15″.

The technical reference field is that related to neuromotor rehabilitation, in which the availability of devices capable of supporting the patient during the neuromotor recovery phases is of considerable utility. Even further, considering the portability of the device 1, it lends itself well to also being used in contexts outside the hospital and in tele-rehabilitation, allowing an autonomous use by the patient.

Obviously, in order to meet contingent and specific needs, those skilled in the art may make further changes and variations to the rehabilitation device 1 according to the present invention, all falling within the scope of protection of the invention, as defined by the following claims.

### REFERENCE NUMERALS

1 rehabilitation device
2 connecting base
3 connecting members
3.1 clamp
3.2 tightening screw
3.3 adhesive surface
3.4 suction cup
3.5 magnet
3.6 Velcro
4 support
5 main body
6 adjustable joint
7′, 7″ first motor, second motor
8′, 8″ first rotary interface, second rotary interface
9′, 9″ first position detecting means, second position detecting means,
10′, 10″ first torque detecting means, second torque detecting means,
11 electronic control device
12 set of grip accessories
12.1 handlebar accessory
12.2 wheel accessory
12.3 crank accessory
12.4 linkage accessory
12.5 translational accessory
13 grip portion
14 coupling portion
15′, 15″ first, second rotation axis
16 connecting seat
17 joint fastening portion
18 base portion of the adjustable joint
19 auxiliary adjustment axis
20 rotation pin
21 tightening bolt
22 outer housing
23′, 23″ first, second transmission
24 adjustable portion of the adjustable joint
25 support seat of the main body
26 main adjustment axis
27 handlebar orientation axis
28 orientation and locking mechanism
29 button
30 motion conversion mechanism
31 anti-rotation means
32 user interface
33 display
34 keyboard
35 electromyographic sensor
36 functional electrical stimulation device
37 EEG signal detector
38 auditory user interface,
39 EDA (electrodermal activity) sensor
40 ECG (electrocardiac activity) sensors
41 digital camera

1. A device for bilateral rehabilitation of a user's limbs, comprising:
a connecting base for a stationary connection of the connecting base to a support,
a main body connected with the connecting base,
a first motor and a second motor supported by the main body,
a first rotary interface supported by the main body in a first position, and which is rotatably operable about a first rotation axis by the first motor, and
a second rotary interface supported by the main body in a second position spaced from the first position and which is rotatably operable about a second rotation axis by the second motor,
a set of grip accessories each of the grip accessories having a grip portion and a coupling portion which are reversibly couplable and integral in rotation to one of the first and second rotary interfaces, respectively,
an electronic control device in signal connection with the first motor and with the second motor and comprising:
first position detecting means for detecting an angular position of the first rotary interface with respect to the main body,
second position detecting means for detecting an angular position of the second rotary interface with respect to the main body,
wherein the electronic control device is configured to control rotary movements applied to the first and second rotary interfaces depending on the angular positions detected by the first and second position detection means,
wherein the main body is connected with the connecting base by an adjustable and lockable joint which allows an adjustment and locking of the position of the main body with respect to the connecting base in at least three different body positions with different orientations of the first and second rotation axes.

2. A device according to claim 1, wherein the electronic control device further comprises:
first torque detecting means for detecting a torque applied to the first rotary interface,
second torque detecting means for detecting a torque applied to the second rotary interface,
wherein the electronic control device is configured to control the rotary movements and torques applied to the first and second rotary interfaces depending on the angular positions detected by the first and second position detecting means and/or the torques detected by the first and second torque detecting means.

3. A device according to claim 1, wherein the connecting base comprises a “U” or clamp shape defining a connecting seat which is laterally accessible to receive an edge of a table.

4. A device according to claim 1, wherein the connecting base forms a joint fastening portion for fastening a base portion of the adjustable joint to the connecting base, and for rotary adjustment of the base position with respect to the connecting base about a horizontal auxiliary adjustment axis.

5. A device according to claim 1, wherein the main body comprises an outer housing which accommodates the first motor and the second motor, in which:
the first motor and the second motor are arranged parallel to each other and side by side, with axial extensions of the first motor and the second motor overlapping, so that an axial dimension of both the first and second motors is less than a sum of the individual axial extensions thereof,
a first operating connection of the first motor to the first rotary interface and a second operating connection of the second motor to the second rotary interface are positioned on opposite sides of the first and second motors, and
the first operating connection is positioned laterally next to the second motor with the axial extensions of the first motor and the second motor overlapping, and the second operating connection is positioned laterally next to the first motor with the axial extensions of the first motor and the second motor overlapping,
the first rotary interface and the second rotary interface are both in laterally displaced positions with respect to both the first motor and the second motor.

6. A device according to claim 1, wherein the first and second rotary interfaces are accessible from outside of the main body and facing two opposite directions towards the outside of the main body.

7. A device according to claim 1, wherein the first and second rotary interfaces are positioned on two opposite sides of the main body and the first rotation axis and the second rotation axis are parallel or coincident.

8. A device according to claim 1, wherein the first and second rotary interfaces comprise quick coupling means, without aid of tools, or one of:
an elastic snap-fit coupler,
a threaded ring nut coupler,
a bayonet coupler,
an insertion coupler,
a threaded coupler,
a rotationally locked connection between the rotary interface and the coupling portion of the grip accessory.

9. A device according to claim 1, wherein the adjustable joint is configured so that the main body is rotatable with respect to the connecting base about a main adjustment axis and lockable with respect to the connecting base,
wherein the main adjustment axis is inclined with respect to the first rotation axis and with respect to the second rotation axis.

10. A device according to claim 9, wherein the main adjustment axis is inclined with respect to the rotation axes at an angle between 45° and 65°, to allow, by a rotary adjustment about only the main adjustment axis, the orientation of the first and second rotation axes in the space along three directions orthogonal to each other.

11. A device according to claim 1, wherein the grip accessories comprise two handlebar accessories, wherein each handlebar accessory is able to couple to one of the first and second rotary interfaces, respectively,
wherein the grip portion is orientable and lockable in position with respect to the coupling portion, about a handlebar orientation axis orthogonal to the first rotation axis and the second rotation axis with the handlebar accessory coupled to the rotary interface.

12. A device according to claim 1, wherein the grip accessories comprise a wheel accessory couplable to one of the first and second rotary interfaces and having two grip portions spaced apart and positioned on two opposite sides with respect to the first or second rotary interface, and adjustable and lockable in position with respect to the coupling portion, around respective handlebar orientation axes.

13. A device according to claim 1, wherein the grip accessories comprise two crank accessories, each of the grip accessories being coupled respectively to one of the first and second rotary interfaces, wherein the grip portion is parallel and spaced with respect to the first rotation axis and with respect to the second rotation axis.

14. A device according to claim 1, wherein the grip accessories comprise a linkage accessory having two said coupling portions both connected to a single grip portion by two articulated linkages.

15. A device according to claim 1, wherein the grip accessories comprise a translational accessory, the grip portion of which is linearly translatable with respect to the coupling portion, and which comprises a non-automatically locking motion conversion mechanism interposed between the grip portion and the coupling portion, as well as anti-rotation means which prevent a rotation of the grip portion with respect to the main body.

16. A device according to claim 1, wherein the coupling portions of the grip accessories form a sure angular reference for a connection with the rotary interface in a single relative angular position.

17. A device according to claim 1, wherein the control device:
comprises a computer connectable to a user interface with a display,
is configured to allow selection of a plurality of rehabilitation programs and adjusting a plurality of movement parameters of the respective rehabilitation program,
wherein the rehabilitation programs comprise rehabilitation programs with movements of the first rotary interface and the second rotary interface which are not synchronized with each other.

18. A device according to claim 1, wherein the control device is configured to perform:
rehabilitation programs with a control of the first motor and the second motor by an angular position control,
rehabilitation programs with a control of the first motor and the second motor by a rotary speed control.

19. A device according to claim 1, wherein the control device is configured to perform rehabilitation programs with a control of the first motor and the second motor by an impedance and/or admittance control, based on:
angular positions of the first rotary interface and of the second rotary interface detected by the first position detecting means and second position detecting means,
theoretical angular positions of the first rotary interface and of the second rotary interface controlled by the control device, and
the corresponding torques transmitted to the first rotary interface and to the second rotary interface, detected by the first torque detecting means and second torque detecting means.

20. A device according to claim 1, wherein the control device is configured for a control of movement of the first motor and to the second motor in a dependent manner, as a function of one or more dependency parameters and dependency functions, which are predefined or selectable by a user interface.

21. A device according to claim 1, wherein the control device is in signal communication with one or more of:
one or more electromyographic sensors,
one or more functional electrical stimulation devices,
one or more EEG signal detectors,
one or more electronic graphical and/or auditory user interfaces,
one or more EDA sensors,
one or more ECG sensors,
one or more digital cameras,
and is configured to control the first motor and the second motor depending on signals provided thereby.

22. A device according to claim 1, wherein both the first and second rotation axes have a fixed relative position therebetween.
